(19) United States
(12) Patent Application Publication
(10) Pub. No.: US 2012/0150230 A1
(43) Pub. Date: Jun. 14, 2012

(54) CROSS CONNECTOR WITH CENTRAL HUB

(75) Inventors: Brent A. Felix, Sandy, UT (US); Eric W. Hooley, North Logan, UT (US); Ronald K. Groberg, Clearfield, UT (US); Timothy R. Maher, Woods Cross, UT (US)

(73) Assignee: INNOVASIS, INC., Salt Lake City, UT (US)

(21) Appl. No.: 13/308,237

(22) Filed: Nov. 30, 2011

Related U.S. Application Data
(60) Provisional application No. 61/421,471, filed on Dec. 9, 2010.

(51) Int. Cl. A61B 17/70

(52) U.S. Cl. 606/250

(57) ABSTRACT

A spinal implant cross connector includes a central hub. Four spaced apart arm assemblies outwardly project from the central hub, each arm assembly having an adjustable length extending between a first end coupled to the central hub and an opposing second end. The arm assemblies extend in a substantially X-shaped pattern with two of the arm assemblies being pivotable relative to the other two. A rod clamp assembly is disposed at the second end of each arm assembly.

### CROSS-REFERENCE TO RELATED APPLICATIONS

This application claims the benefit of Provisional Application Ser. No. 61/421,471, filed Dec. 9, 2010, which application is incorporated herein by specific reference.

### BACKGROUND OF THE INVENTION

1. The Field of the Invention

The present invention relates to cross connectors for use in association with spine stabilizing systems and, more specifically, for connecting together adjacent rods of spine stabilizing systems.

2. The Relevant Technology

Various spinal stabilizing systems are available for adjusting or fixing adjacent vertebrae of the spine relative to each other. For example, such systems can be used when it is necessary to fuse together two adjacent vertebrae. In conventional procedures, adjacent vertebrae are stabilized by securing a first pair of polyaxial screws to a first vertebrae. The polyaxial screws are secured on opposing lateral sides of the vertebrae. A second pair of polyaxial screws is then secured to an adjacent vertebrae on the opposing sides thereof. As needed, additional pairs of polyaxial screws can be secured on the opposing sides of further consecutive vertebrae.

Once the polyaxial screws are positioned, an elongated first rod is secured to each of the polyaxial screws on a first side while an elongated second rod is secured to each of the polyaxial screws on the opposing second side. The rods help to secure each of the vertebrae in a fixed location relative to the others. To help stabilize lateral movement of the vertebrae, a plurality of cross connectors can be connected between the first rod and the second rod at spaced apart locations along the length of the rods. A conventional cross connector comprises a linear shaft having a clamp formed on each end. The clamps are designed to selectively couple with the first and second rods. Although conventional cross connectors are effective, they often have a rigid structure making them difficult to install, difficult to adjust, and/or difficult to secure in place. Conventional cross connectors can also permit unwanted movement of the adjacent rods due to the high torsion and other forces applied by the rods on the cross connectors.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 is a perspective view of an inventive cross connector coupled to a pair of rods;

FIG. 2 is top perspective view of the cross connector shown in FIG. 1;

FIG. 3 is a bottom perspective view of the cross connector shown in FIG. 1;

FIG. 4 is a partially exploded view of the cross connector shown in FIG. 1;

FIG. 5 is an exploded bottom view of the rod clamp assembly show in FIG. 4;

FIG. 6 is a cross sectional side view of the rod clamp assembly shown in FIG. 4;

FIG. 7 is a partially exploded view of and alternative embodiment of the central hub shown in FIG. 4; and

FIG. 8 is a perspective view of an alternative embodiment of the arm assemblies shown in FIG. 4.

### DETAILED DESCRIPTION OF THE PREFERRED EMBODIMENTS

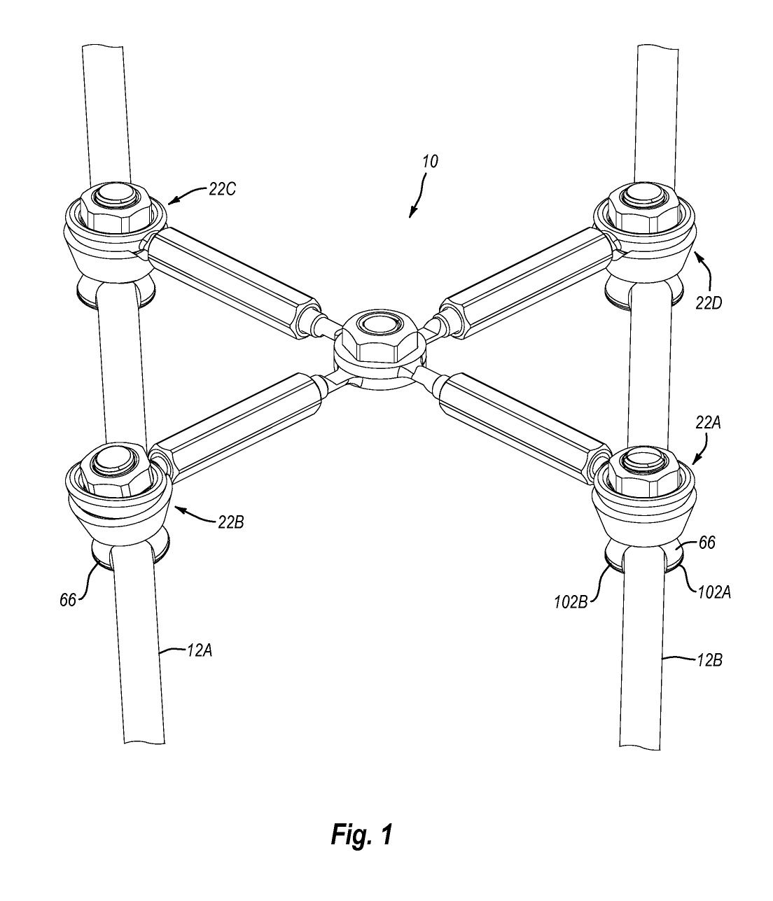

Depicted in FIG. 1 is one embodiment of a cross connector 10 incorporating features of the present invention. Cross connector 10 is shown being used in association with a first rod 12A and an opposing second rod 12B which can comprise spine stabilizing rods. Rods 12A and 12B can in turn be connected to a series of pedicle screws, polyaxial screws, or other fasteners that are mounted on adjacent vertebrae of the spine. Cross connector 10 functions to secure rods 12A and 12B relative to each other, thereby securing the adjacent vertebrae relative to each other. In view of the forgoing, cross connector 10 can function as part of a spinal implant or spine stabilizing system.

In alternative embodiments, however, it is appreciated that cross connector 10 need not be used in association with the spine but can be used in other procedures where it is necessary to stabilize adjacent rods. It is also appreciated that cross connector 10 can be used in association with a variety of different spine stabilizing systems.

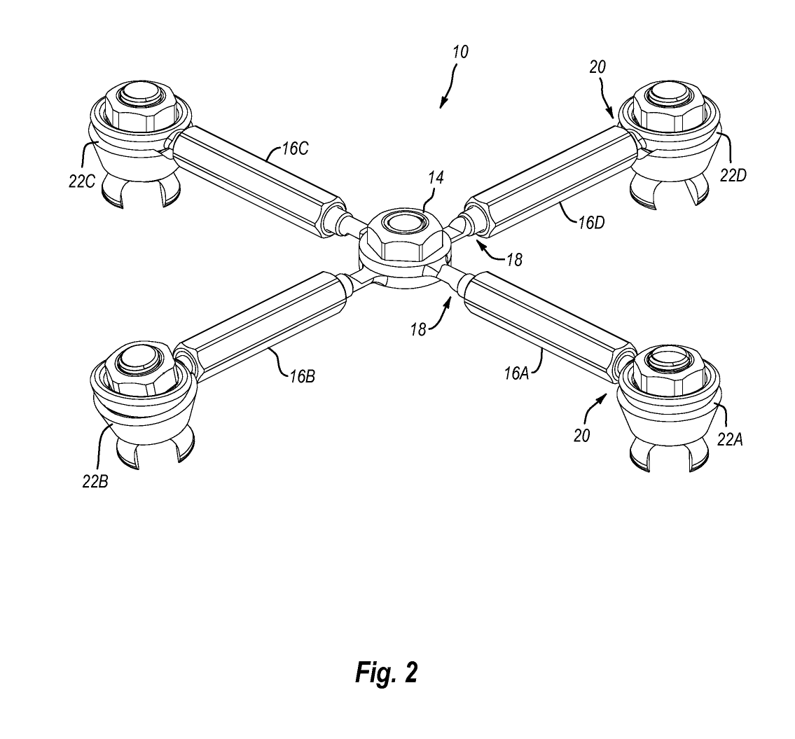

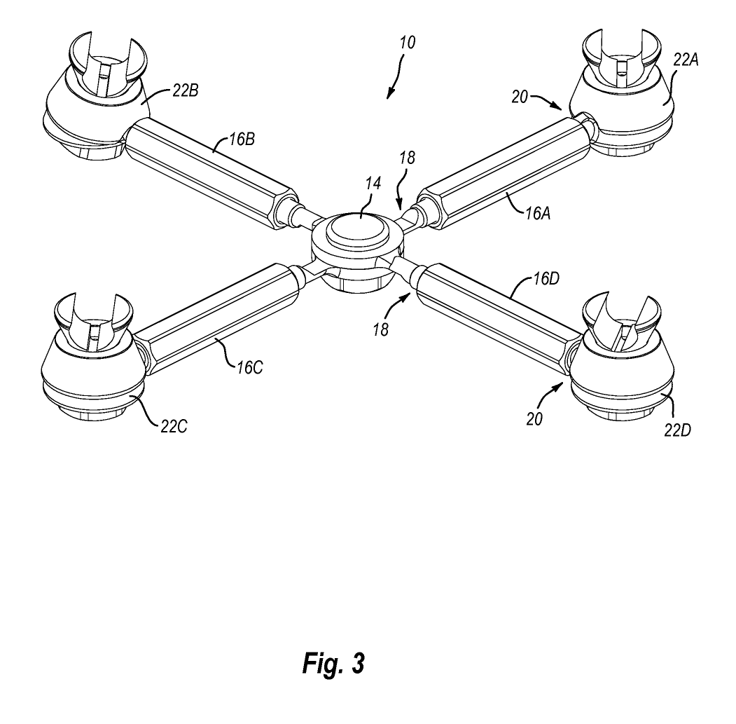

As depicted in FIGS. 2 and 3, cross connector 10 generally comprises a central hub 14 having four arm assemblies 16A-D radially outwardly projecting from hub 14. Each arm assembly 16A-D has a first end 18 secured to central hub 14 and an opposing second end 20. Mounted on second end 20 of each arm assembly 16A-D is a corresponding rod clamp assembly 22A-D. As will be discussed below in greater detail, each arm assembly 16A-D is adjustable along its length so that each rod clamp assembly 22 can be adjusted closer to or further away from hub 14. As will also be discussed below in greater detail, arm assemblies 16A-D are disposed in a substantially X-shaped configuration. In one embodiment, arm assemblies 16A and 16C are rigidly secured together and arm assemblies 16B and 16D are rigidly secured together with arm assemblies 16A and 16C being free to pivot relative to arm assemblies 16B and 16D about central hub 14. That is, cross connector 10 can expand and collapse using a scissor like movement.

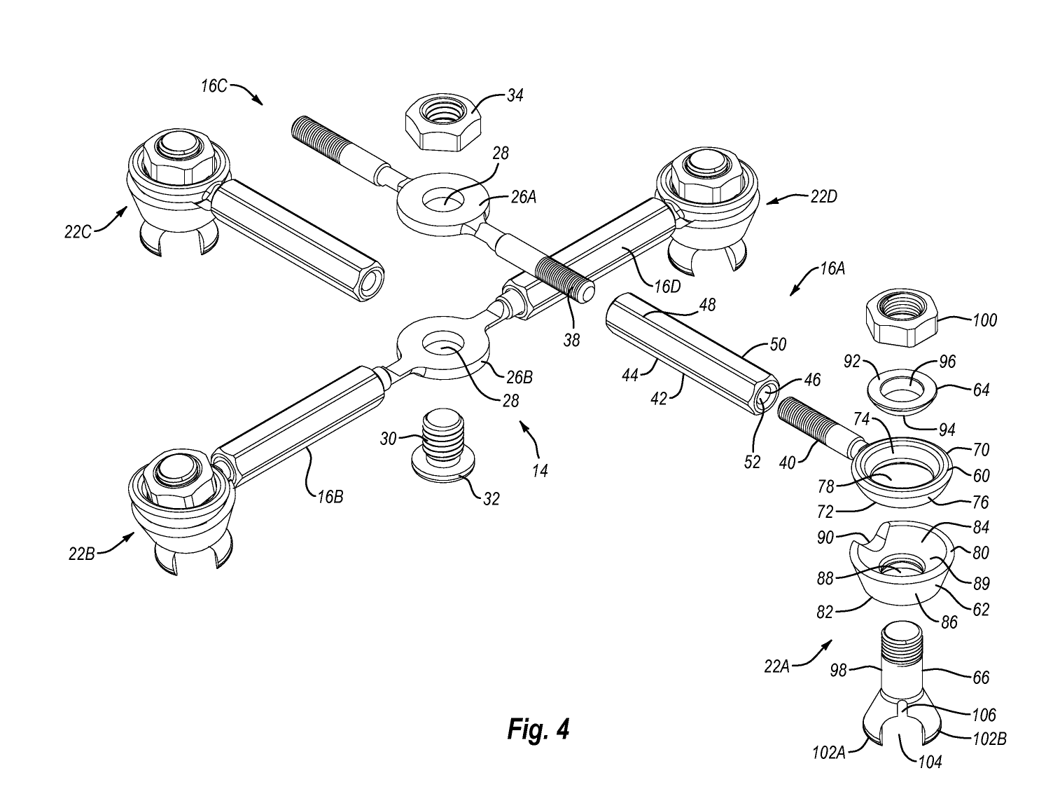

Turning to FIG. 4, hub 14 is shown as comprising a first support 26A and a second support 26B each having a circular disk-shaped configuration with an opening 28 extending therethrough. The openings 28 of support 26A and B are aligned and a fastener 30 extends therethrough. Fastener 30 holds supports 26A and B together but permits supports 26A and B to pivot relative to each other about a central longitudinal axis of openings 28 and/or fastener 30. In the embodiment depicted, fastener 30 comprises a bolt 32 that engages with a nut 34. In alternative embodiments, bolt 32 and nut 34 can be replaced by a rivet or any other type of fastener that secures but permits relative rotation of supports 26A and B. Although not required, in one embodiment fastener 30 can be designed so that the surgeon can selectively tighten fastener 30 and thereby more tightly secure together supports 26A and 26B together.

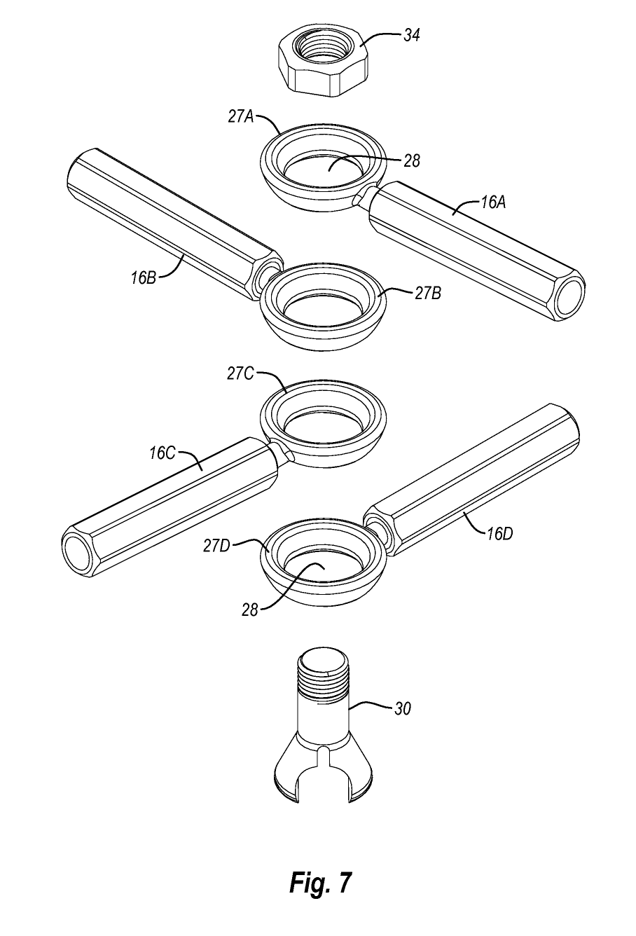

Arm assemblies 16A and 16C project from opposing sides of first support 26A while arm assemblies 16B and 16D project from opposing sides of first support 26B. In an alternative embodiment, each arm assembly 16A-D can project from a separate support 26 with all four supports being secured together by fastener 30. For example, as depicted in FIG. 7, supports 26A and 26B can be replaced with supports 27A-D each having a circular disk-shaped configuration with an opening 28 extending therethrough. Arm assembly 16A-D radially outwardly project from a corresponding one of supports 27A-D, respectively. The openings 28 of supports 27A-D are aligned and fastener 30 can extend therethrough to hold supports 27A-D together and permit supports 27A-D to pivot relative to each other about a central longitudinal axis of openings 28 and/or fastener 30.

In one embodiment, each arm assembly 16A-D comprises a turnbuckle. More specifically, as depicted in FIG. 4, arm assembly 16A comprises a threaded first shaft 38 radially outwardly projecting from a perimeter edge of first support 26A and includes a threaded second shaft 40 radially outwardly projecting from rod clamp assembly 22A. Arm assembly 16A further includes a tubular collar 42 having an exterior surface 44 and an interior surface 46 that each extend between a first end 48 and an opposing second end 50. Interior surface 46 forms a threaded bore 52 passing through collar 42. First shaft 38 is threaded into bore 52 at first end 48 while second shaft 40 is threaded into bore 52 at second end 50. Shafts 38 and 40 are threaded such that as collar 42 is manually rotated, shafts 38 and 40 either concurrently advance into collar 42 or concurrently advance out of collar 42, thereby enabling selective positioning of rod clamp assembly 22A. To that end, the threads on shafts 38 and 40 can be reversed. To help facilitate manual rotation of collar 42, the exterior surface of collar 42 can have a polygonal or other non-circular transverse cross section that permits easy gripping. A texture can also be formed on the exterior surface.

Arm assemblies 16B-D can have the same configuration as arm assembly 16A and thus like reference numbers are used to identify like reference characters.

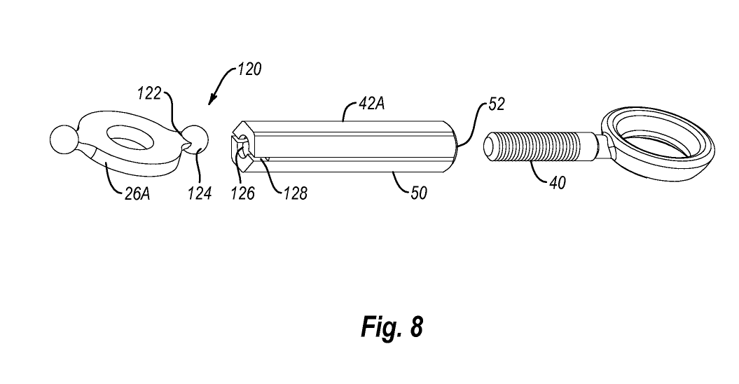

In an alternative embodiment, it is appreciated that arm assemblies 16A-D need not comprise of turnbuckle. For example, in one embodiment first shaft 38 can be modified or eliminated and collar 42 can mounted on first support 26A so that it can freely rotate relative thereto. Once specific example of such as assembly is depicted in Figure 8. As show therein, support 26A has an arm 120 radially outwardly projecting from opposing sides of the perimeter edge thereof. Each arm 120 comprises a stem 122 having an enlarged rounded head 124 formed on the end thereof. A collar 42A is shown that has threaded bore 52 formed at second end 50. However, formed at first end 48 is a socket 126 having a constricted opening. An expansion slot 128 extends through the side of collar 42A and communicates with socket 126. Socket 126 is configured so that enlarged head 124 can be press fit into and captured within socket 126. Expansion slot 128 permits resilient expansion of the constricted opening so that enlarged head 124 can be received within socket 126. Depending on sizing and material selection, expansion slot 128 can be eliminated.

In the assembled configuration, rotation of collar 42A causes collar 42A to freely spin on enlarged head 124. However, depending on the direction of rotation of collar 42A, second shaft 40 advances into or out of collar 42A. In another alternative embodiment, first shaft 38 can be used but second shaft 40 can be replaced with arm 120. Collar 42A can then be inverted 180 degrees and coupled with first shaft 38 and arm 120. Rotation of collar 42A then cause first shaft 38 to advance into or out of collar 42 while collar 42A freely spins relative to rod clamp assembly 22A. Arm assemblies 16B-D can be likewise configured. It is appreciated that arm 120 and socket 126 can be replaced by other structures that perform the same function such as a union or slip joint.

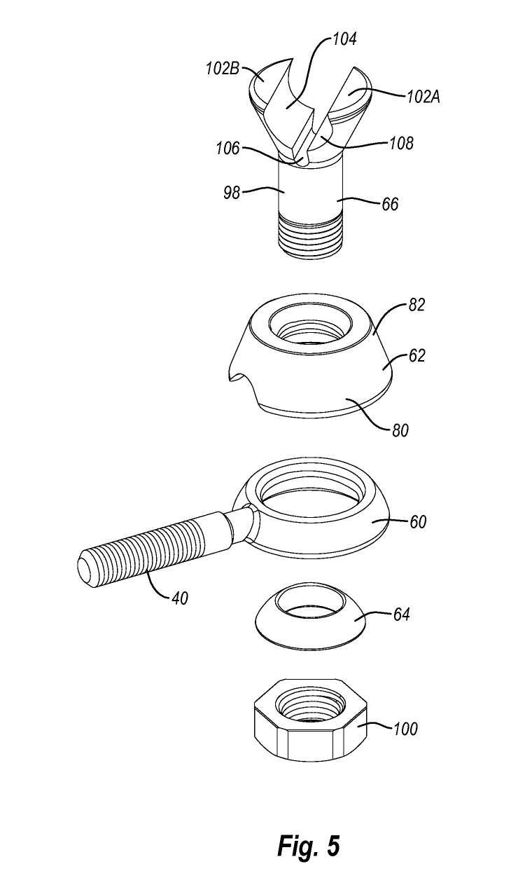

Returning to FIGS. 4 and 5, rod clamp assembly 22A comprises an annular seat 60, a annular swivel 62 that receives seat 60, a washer 64 that is disposed on seat 60, and a clamp 66 that passes through seat 60, swivel 62, and washer 64, and that engages with a nut 100. More specifically, second shaft 40 radially outwardly projects from the side of seat 60. Seat 60 has a first end 70 and an opposing second end 72 with an interior surface 74 and an exterior surface 76 extending therebetween. An opening 78 passes centrally down through seat 60 between first end 70 and second end 72. Interior surface 74 and exterior surface 76 both radially inwardly taper as they extend from first end 70 to second end 72.

Swivel 62 similarly has a first end 80 and an opposing second end 82 with an interior surface 84 and an opposing exterior surface 86 extending therebetween. A rounded notch 90 is formed on first end 80 of swivel 62 to receive second shaft 40. An opening 88 centrally passes down through swivel 62 from first end 80 to second end 82. Exterior surface 86 radially inwardly tapers as it extends from first end 80 to second end 82.

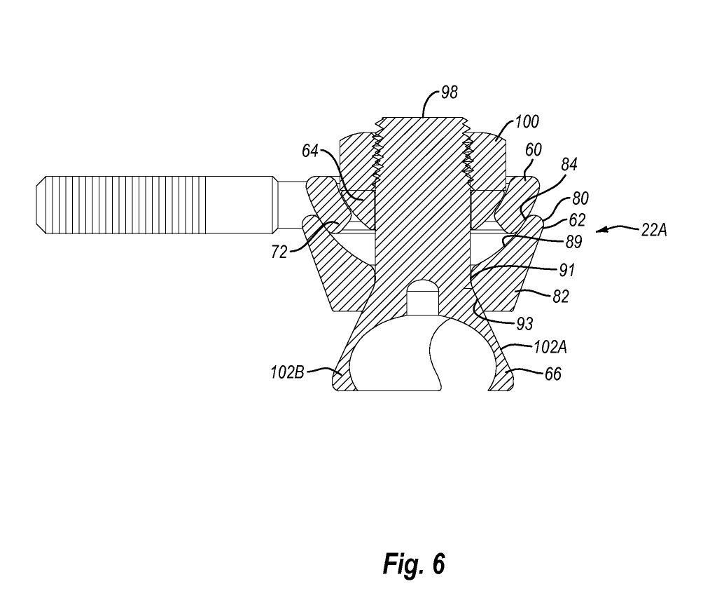

As perhaps best depicted in FIG. 6, interior surface 84 includes an upper recess 89 that radially inwardly tapers from first end 80 down to a neck 91. Interior surface 84 also includes a lower recess 93 that radially inwardly tapers from second end 82 up to neck 91. Upper recess 89 of swivel 62 is configured to receive second end 72 of seat 60 so that swivel 62 can freely pivot on seat 60. In one embodiment, the taper of upper recess 89 of swivel 62 has a configuration complimentary to the taper on exterior surface 76 of seat 60 to help facilitate free sliding between the members.

Returning to FIG. 4, washer 64 comprises a substantially flat top surface 92 and a bowel shaped bottom surface 94 with an opening 96 centrally extending therebetween. Bottom surface 94 is configured to be received within seat 60 so that washer 64 can freely pivot therein. In this regard, the taper of bottom surface 94 is typically complimentary to the taper on interior surface 74 of seat 60.

When seat 60, swivel 62, and washer 64 are nested together (FIG. 6), openings 78, 88, and 96 extending therethrough are aligned. Clamp 66 comprises a shaft 98 having a threaded end that passes through the aligned openings and engages with a nut 100. In this assembled configuration, clamp 66 and nut 100 can pivot concurrently with swivel 62 and washer 64 relative to seat 60. As shown in FIG. 5, clamp 66 further comprises an engager projecting from the end of shaft 98 that is configured to engage rod 12A or B (FIG. 1). In the embodiment depicted, the engager comprises a pair of clamping arms 102A and B that project from the end of shaft 98 and flare radially outward. A rounded channel 104 is formed between clamping arms 102A and B and is configured to receive rod 12B (FIG. 1). More specifically, each clamping arm 102A and B has an interior surface 108 having a concave curvature that is generally complementary to the curvature of rod 12B.

A notch 106 is formed at the intersection between clamping arms 102A and B to permit arms 102A and B to bend inwardly for clamping against rod 12B. For example, when rod clamp assembly 22A is assembled as shown in FIG. 6, clamping arms 102A and B are received within lower recess 93 of swivel 62. Because lower recess 93 inwardly tapers and clamping arms 102A and B outwardly flare, as nut 100 is tightened, clamp 66 is drawn into lower recess 93 which causes clamping arms 102A and B to inwardly bend and clamp onto rod 12B disposed therebetween (FIG. 1), thereby securing rod 12B to clamp 66. The other rod clamp assemblies 22B-D can have the same configuration as rod clamp assembly 22A and thus like reference characters can be used to identify like elements. In alternative embodiment, the engager can comprise a clamp or other type of fastener for engaging with rod 12.

The elements of cross connector 10 can be comprised of titanium, stainless steel, carbon reinforced composites, PEEK and other biocompatible materials having required strength properties. It is also appreciated that different parts can be made of different materials depending on desired properties.

During use, as shown in FIG. 1, cross connector 10 is initially freely movable by pivoting clamps 66, lengthening arms assemblies 16A-D and scissoring the X-shaped configuring. By adjusting these features, clamps 66 of rod clamp assemblies 22B and C can be secured to rod 12A while clamps 66 of rod clamp assemblies 22A and D can be secured to rod 12B. The turnbuckles and nuts 34 and 100 can then be tightened so that all of cross connector 10 is rigid, thereby securing and stabilizing rods 12A and B relative to each other.

In view of the foregoing, cross connector 10 has a number of unique properties. For example, each clamp 66 can freely pivot concurrently with swivel 62 relative to seat 60. This pivoting provides greater flexibility when attaching clamp 66 to a rod. The ability to scissor arm assemblies 16A and C relative to arm assemblies 16B and D and to adjust the length of the arm assemblies also facilitates greater adaptability when positioning cross connector 10 and securing it to the rods.

Furthermore, as a result of the X-shaped configuration of cross connector 10, cross connector 10 provides greater stability to rods 12A and B relative to conventional linear cross connectors. That is, the rods 12A and B that are typically disposed in substantially parallel alignment along the vertebra will naturally want to move relative to each other as the patient moves. As a result, the rods can apply torsion and other forces on the cross connectors. By having a cross connector with an X-shaped configuration, the two arm assemblies connected to a specific rod produce a triangular support that has significantly greater resistant to the torsion and other forces applied by the rods relative to conventional cross connectors and thus is better able to stabilize the spine.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A spinal implant cross connector comprising:
a central hub;
four spaced apart arm assemblies outwardly projecting from the central hub, each arm assembly having an adjustable length extending between a first end coupled to the central hub and an opposing second end; and
a rod clamp assembly disposed at the second end of each arm assembly.

2. The spinal implant cross connector as recited in claim 1, wherein the arm assemblies project from the hub so as to have a substantially X-shaped configuration.

3. The spinal implant cross connector as recited in claim 1, wherein the central hub comprises:
a first support having two of the arm assemblies projecting therefrom;
a second support having the other two arm assemblies projecting therefrom; and
a fastener pivotably connecting the first support to the second support.

4. The spinal implant cross connector as recited in claim 1, wherein the central hub comprises:
four separate and distinct supports, each support having a separate arm assembly projecting therefrom; and
a fastener pivotably connecting the four supports together.

5. The spinal implant cross connector as recited in claim 1, wherein each arm assembly comprises a turnbuckle.

6. The spinal implant cross connector as recited in claim 1, wherein each arm assembly comprises:
a collar having a first end and an opposing second end, a threaded passage being formed at the first end;
a threaded shaft secured to the hub or the rod clamp assembly, the shaft being in threaded engagement with the passage of the collar; and
the second end of the collar being coupled with the other of the hub or the rod clamp so that the collar can spin relative to the hub or rod clamp to which it is coupled.

7. The spinal implant cross connector as recited in claim 1, wherein two of the arm assemblies can pivot relative to the remaining two arm assemblies.

8. The spinal implant cross connector as recited in claim 1, wherein each rod clamp assembly comprises:
a tapered seat secured to a turnbuckle and having an opening extending therethrough;
a swivel having a tapered recess in which the seat is positioned, the swivel being movable relative to the seat;
a washer disposed on a side of the seat opposite the swivel; and
a clamp comprising a locking shaft extending through the seat, swivel and washer and an engager configured to engage a rod.

9. The spinal implant cross connector as recited in claim 1, wherein the engager comprises a pair of clamping arms projecting from the locking shaft, the clamping arms bounding a channel therebetween, the channel being configured to receive a rod.

10. The spinal implant cross connector as recited in claim 1, further comprising:
a first rod removably coupled to two of the rod clamp assemblies; and
a second rod removably coupled to the other two of the rod clamp assemblies.

11. A spinal implant cross connector comprising:
a central hub;
a plurality of spaced apart turnbuckles outwardly projecting from the central hub, each turnbuckle having a first end coupled to the central hub and an opposing second end; and
a rod clamp assembly disposed at the second end of each turnbuckle.

12. The spinal implant cross connector as recited in claim 11, wherein the turnbuckles project from the hub so as to have a substantially X-shaped configuration.

13. The spinal implant cross connector as recited in claim 11, wherein the central hub comprises:
a first support having two of the turnbuckles projecting therefrom;
a second support having the other two turnbuckles projecting therefrom; and
a fastener pivotably connecting the first support to the second support.

14. The spinal implant cross connector as recited in claim 11, wherein each rod clamp assembly comprises:
a tapered seat secured to a turnbuckle and having an opening extending therethrough;
a swivel having a tapered recess in which the seat is positioned, the swivel being movable relative to the seat;
a washer disposed on a side of the seat opposite the swivel; and
a clamp comprising a locking shaft extending through the seat, swivel and washer and an engager configured to engage a rod.

15. The spinal implant cross connector as recited in claim 14, wherein the engager comprises a pair of clamping arms projecting from the locking shaft, the clamping arms bounding a channel therebetween, the channel being configured to receive a rod.
